(19) United States
(12) Patent Application Publication
(10) Pub. No.: US 2026/0272340 A1
(43) Pub. Date: Sep. 17, 2026

(54) Sensor Device

(71) Applicant: Taiyo Yuden Co., Ltd., Tokyo (JP)

(72) Inventors: Yuki SAITO, TOKYO (JP); Katsuhiro OYAMA, TOKYO (JP)

(21) Appl. No.: 19/567,894

(22) Filed: Mar. 16, 2026

(30) Foreign Application Priority Data
Mar. 17, 2025 (JP) 2025-042960

(51) Int. Cl. A61B 5/1455; A61B 5/00; A61B 5/145

(52) U.S. Cl. CPC A61B 5/1455; A61B 5/14532; A61B 5/6824; A61B 5/6831

(57) ABSTRACT

A sensor device includes a light-emitting element, a prism that has a first bottom surface for contact with a surface of a human body, a second bottom surface lying parallel to the first bottom surface, and multiple slanted surfaces and refracts light toward the human body, a first light-detecting element detecting light having been reflected by a region in the human body, refracted by the prism, and emitted from the prism, a second light-detecting element detecting light having been reflected the first bottom surface of the prism into the prism and emitted from the prism, an estimating section estimating a concentration of a biological component included in the human body from an output from the first light-detecting element, and a determining section determining whether the estimating section can estimate the concentration of the biological component on the basis of at least an output from the second light-detecting element.

### BACKGROUND

The present disclosure relates to a sensor device.

In fields of medical care and health care, it has been customary to examine a concentration of a biological component contained in human bodies, e.g., a concentration of glucose in blood, by taking blood samples from the human bodies. However, taking blood samples from patients occurs with concerns about physical and mental burdens such as pain, discomfort, and anxiety of the patients and concerns about infectious diseases.

Consequently, research efforts have been made to use light such as near-infrared light to obtain a noninvasive estimate of the concentration of a biological component (see, for example, JP 2023-15889A and JP 2019-42500A).

### SUMMARY

According to the noninvasive optical measurement technology, light reflected or scattered by a region inside a human body is measured. Therefore, of light emitted from a light-emitting element, as much light reflected or scattered by a region inside a human body as possible should preferably be applied to a light-detecting element.

One approach is to use a sensor device having a prism that can be held in surface contact with the surface of a human body for refracting light emitted from a light-emitting element toward the human body and reflecting light reflected or scattered by a region inside the human body toward a light-detecting element.

However, even though the prism and the surface of the human body are held in surface contact with each other, infinitesimal spaces may possibly be present between the prism and the surface of the human body. If such infinitesimal spaces exit, since the refractive index of the prism, the refractive index of air, and the refractive index of the surface of the human body are different from each other, the interface between the prism and air tends to refract and reflect the light in directions other than desired directions. The larger the infinitesimal spaces are, the larger the amount of light reflected by the interface becomes, resulting in a smaller amount of light that is reflected or scattered by the region in the human body and applied to the light-detecting device and hence in a lower level of accuracy of the estimate of the concentration of a biological component in the human body.

Consequently, it is desirable to refrain from obtaining an estimate of the concentration of a biological component in a human body if an infinitesimal space present between the surface of the human body and a prism held in surface contact therewith is small in size, or stated otherwise, the degree of close contact between the surface of the human body and the prism held in surface contact therewith is low.

It is desirable to provide a sensor device that refrains from obtaining an estimate of the concentration of a biological component in a human body if the degree of close contact between the surface of the human body and a prism of the sensor device is not sufficient and that obtains an estimate of the concentration of the biological component in the human body if the degree of close contact between the surface of the human body and the prism is sufficient.

In accordance with an aspect of the present disclosure, there is provided a sensor device including a light-emitting element, a prism, a first light-detecting element, a second light-detecting element, an estimating section, and a determining section. The prism is of a trapezoidal contour as viewed along a direction and has a first bottom surface for contact with a surface of a human body, a second bottom surface lying parallel to the first bottom surface, and multiple slanted surfaces joining the first bottom surface and the second bottom surface to each other. The prism refracts light emitted from the light-emitting element and applied to a first slanted surface among the multiple slanted surfaces toward the human body. The first light-detecting element detects, of the light emitted from the light-emitting element, light that has been reflected by a region in the human body, refracted by the prism, and emitted from the prism. The second light-detecting element detects, of the light emitted from the light-emitting element, light that has been reflected by the first bottom surface of the prism into the prism and emitted from the prism. The estimating section that estimates a concentration of a biological component included in the human body from an output from the first light-detecting element. The determining section determines whether the estimating section can estimate the concentration of the biological component or not on the basis of at least an output from the second light-detecting element.

According to the aspect of the present disclosure, the second light-detecting element is provided in addition to the first light-detecting element that is used to measure the light, of the light emitted from the light-emitting element, reflected by the region in the human body and estimate the concentration of the biological component. The second light-detecting element measures the light, of the light emitted from the light-emitting element, reflected by the first bottom surface of the prism into the prism and emitted from the prism. If the output from the second light-detecting element is large, the degree of close contact between the surface of the human body and the prism is low, and if the output from the second light-detecting element is small, the degree of close contact between the surface of the human body and the prism is high. The larger the output from the second light-detecting element is, the smaller the amount of light detected by the first light-detecting element is. It is determined whether the estimating section can estimate the concentration of the biological component or not on the basis of the output from the second light-detecting element. If the degree of close contact between the surface of the human body and the prism is insufficient, the estimating section does not estimate the concentration of the biological component, and if the degree of close contact between the surface of the human body and the prism is sufficient, the estimating section estimates the concentration of the biological component.

The determining section may decide that the estimating section can estimate the concentration of the biological component if the magnitude of the output from the second light-detecting section is equal to or smaller than the first threshold value. The determining section may decide that the estimating section is not allowed to estimate the concentration of the biological component if the magnitude of the output from the second light-detecting section is larger than the first threshold value.

The sensor device may further include an indicating section that gives an indication to a user of the sensor device. The determining section may cause the indicating section to issue a request for prompting the user to press the prism strongly against the human body if the magnitude of the output from the second light-detecting section is larger than the first threshold value.

The determining section may cause the indicating section to indicate that the estimating section should cancel its operation to estimate the concentration of the biological component and may cause the estimating section to cancel the operation if the magnitude of the output from the second light-detecting section is equal to or smaller than the first threshold value after elapse of a predetermined period of time from an initial event of detection that the magnitude of the output from the second light-detecting element is larger than the first threshold value.

In this case, the sensor device may further include a pressing mechanism that presses the prism against the human body and a controlling section that controls the pressing mechanism. When the magnitude of the output from the second light-detecting section exceeds the first threshold value, the controlling section may control the pressing mechanism to increase its pressing force until the magnitude of the output from the second light-detecting section becomes equal to or smaller than the first threshold value, and after the magnitude of the output from the second light-detecting section has become equal to or smaller than the first threshold value, the controlling section may maintain the pressing force. The estimating section is allowed to estimate the concentration of the biological component from the output from the first light-detecting element under the maintained pressing force.

The controlling section may drive the pressing mechanism in a first mode during an initial stage of its control over the pressing mechanism and after the initial stage, may drive the pressing mechanism in a second mode that is of a lower rate than the first mode to increase the pressing force from the pressing mechanism until the magnitude of the output from the second light-detecting section becomes equal to or smaller than the first threshold value.

The determining section may decide that the estimating section can estimate the concentration of the biological component if a ratio of the magnitude of the output from the second light-detecting element to the magnitude of the output from the first light-detecting section is equal to or smaller than a second threshold value and that the estimating section is not allowed to estimate the concentration of the biological component if the ratio is larger than the second threshold value.

In this case, the sensor device may further include an indicating section that gives an indication to the user of the sensor device. The determining section may cause the indicating section to issue a request for prompting the user to press the prism strongly against the human body if the ratio is larger than the second threshold value.

The determining section may cause the indicating section to indicate that the estimating section should cancel its operation to estimate the concentration of the biological component and cause the estimating section to cancel the operation if the ratio is equal to or smaller than the second threshold value after elapse of a predetermined period of time from an initial event of detection that the ratio is larger than the second threshold value.

The sensor device may further include a pressing mechanism that presses the prism against the human body and a controlling section that controls the pressing mechanism. When the ratio exceeds the second threshold value, the controlling section may control the pressing mechanism to increase its pressing force until the ratio becomes equal to or smaller than the second threshold value, and after the ratio has become equal to or smaller than the second threshold value, the controlling section may maintain the pressing force. The estimating section is allowed to estimate the concentration of the biological component from the output from the first light-detecting element under the maintained pressing force.

In this case, the controlling section may drive the pressing mechanism in a first mode during an initial stage of its control over the pressing mechanism and after the initial stage, may drive the pressing mechanism in a second mode that is of a lower rate than the first mode to increase the pressing force from the pressing mechanism until the ratio becomes equal to or smaller than the second threshold value.

The first light-detecting element may be disposed to detect light emitted from the second bottom surface. The second light-detecting element may be disposed to detect light emitted from a second slanted surface among the slanted surfaces that is disposed opposite the first slanted surface across the second bottom surface.

Alternatively, the first light-detecting element may be disposed to detect light emitted from the second bottom surface. The second light-detecting element may be disposed to detect light emitted from the second bottom surface.

In this case, the second bottom surface of the prism may have a central area and a peripheral area disposed in the periphery of the central area. The first light-detecting element may be disposed in facing relation to the central area and the second light-detecting element may be disposed in facing relation to the peripheral area.

Alternatively, the second light-detecting element may have an area larger than the first light-detecting element and may have an overlapping portion overlapping the first light-detecting element and a protrusive portion protruding from the first light-detecting element, the protrusive portion being disposed in facing relation to the peripheral area.

The sensor device may have multiple light-emitting elements and multiple the second light-detecting elements.

The light-emitting element may emit near-infrared light in a wavelength range of 780 to 2500 nm.

According to the aspect of the present disclosure, the sensor device does not obtain an estimate of the concentration of the biological component in the human body if the degree of close contact between the surface of the human body and the prism is not sufficient and obtains an estimate of the concentration of the biological component in the human body if the degree of close contact between the surface of the human body and the prism is sufficient.

### BRIEF DESCRIPTION OF THE DRAWINGS

FIG. 1 is a front elevational view of a sensor device according to an embodiment of the present disclosure as it is in use on a human body;

FIG. 2 is a cross-sectional view of a part of the sensor device and the human body, illustrating the principles of the sensor device for obtaining an estimate of the concentration of a biological component in the human body;

FIG. 3 is a plan view of a part of the sensor device according to the embodiment;

FIG. 4 is a plan view of a part of a sensor device according to a modification of the embodiment;

FIG. 5 is a plan view of a part of a sensor device according to another modification of the embodiment;

FIG. 6 is a graph illustrating optical absorption spectrums of oxygenated hemoglobin and reduced hemoglobin;

FIG. 7 is a cross-sectional view illustrating an example of paths of light in the case where the degree of close contact between the surface of a human body and a trapezoidal prism held in surface contact therewith is low;

FIG. 8 is a graph illustrating a relation between a thickness of an air layer between the surface of the human body and the trapezoidal prism and a magnitude of the output from a second light-detecting element;

FIG. 9 is a flowchart of a process of using the sensor device according to the embodiment;

FIG. 10 is a flowchart of another process of using the sensor device according to the embodiment;

FIG. 11 is a front elevational view of a sensor device according to a modification of the embodiment;

FIG. 12 is a block diagram of a control system of the sensor device illustrated in FIG. 11;

FIG. 13 is a flowchart of a process of using the sensor device illustrated in FIG. 11;

FIG. 14 is a flowchart of another process of using the sensor device illustrated in FIG. 11;

FIG. 15 is a cross-sectional view of a part of a sensor device according to a modification of the embodiment and the human body; and

FIG. 16 is a cross-sectional view of a part of a sensor device according to another modification of the embodiment and the human body.

### DETAILED DESCRIPTION OF THE PREFERRED EMBODIMENT

A preferred embodiment of the present disclosure will be described in detail below with reference to the accompanying drawings. A scale of the drawings is not necessarily accurate and some features may be illustrated as exaggerated or omitted from illustration.

Sensor devices according to the embodiment of the present disclosure have components mounted on a part, e.g., an arm, of the body of an examinee such as a patient and are used to measure the concentration of a biological component contained in the body of the examinee.

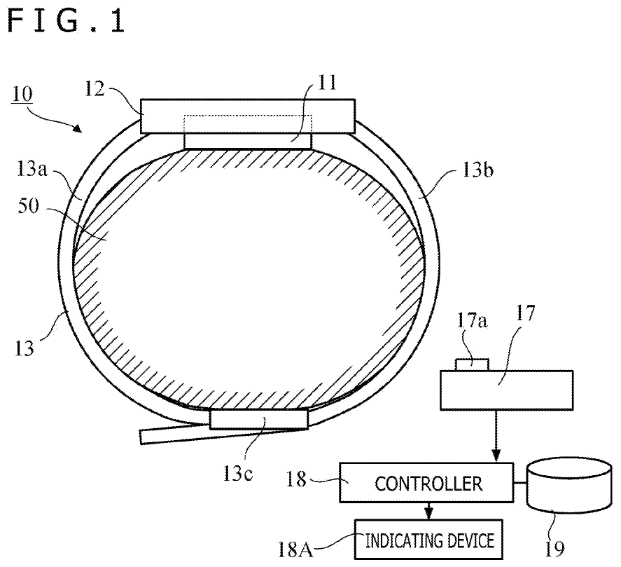

As illustrated in FIG. 1, a sensor device 10 according to the embodiment includes a housing 11, a support 12, a belt 13, a switch unit 17, a controller 18, an indicating device 18A, and a storage device 19.

The housing 11 accommodates therein an optical system of the sensor device 10 as described later.

The support 12 is shaped as a flat plate that supports the housing 11.

The belt 13 has two belt pieces 13a and 13b and a buckle 13c disposed remotely from the support 12. Each of the belt pieces 13a and 13b has an end and an opposite end, and the support 12 has both ends that are joined to the respective ends of the support 12. The buckle 13c secures the respective opposite ends of the belt pieces 13a and 13b. For example, the buckle 13c may have prongs engaging in holes in the belt pieces 13a and 13b, or the buckle 13c may be fixed to the belt pieces 13a and 13b by a hook-and-loop fastener. In use, the belt 13 is wound around an arm 50 of the examinee such that the optical system of the sensor device 10 that is housed in the housing 11 is placed in a stationary position near the arm 50.

Alternatively, the optical system of the sensor device 10 may be disposed near another region of the examinee, such as one of the four limbs, the neck, the head, or the abdominal part of the examinee, with the belt 13 wound around the other region.

The switch unit 17 has a start button 17a. When the examinee or a medical attendant in charge presses the start button 17a, the switch unit 17 supplies the controller 18 with a start command, enabling the sensor device 10 to start measuring the concentration of the biological component of the examinee.

The controller 18 is a computer that operates according to computer programs, for example. The controller 18 is connected to the switch unit 17 through a wireless or wired link for receiving the start command from the switch unit 17. The controller 18 is also connected to the indicating device 18A through a wireless or wired link for supplying a control signal to the indicating device 18A. Moreover, the controller 18 is further connected to the optical system in the housing 11 through a wireless or wired link for supplying a drive signal to the optical system and receiving a measurement signal from the optical system.

As described later, the controller 18 functions as an estimating section for estimating the concentration of a biological component contained in the human body and a determining section for determining whether the estimating section can estimate the concentration of the biological component or not.

The indicating device, which may be referred to as an “indicating section,” 18A gives an indication to a user of the sensor device 10. The user of the sensor device 10 may be the examinee or the medical attendant, and the indicating device 18A may be a display device or a speaker.

The storage device 19 stores data used by the controller 18 and the computer programs run by the controller 18.

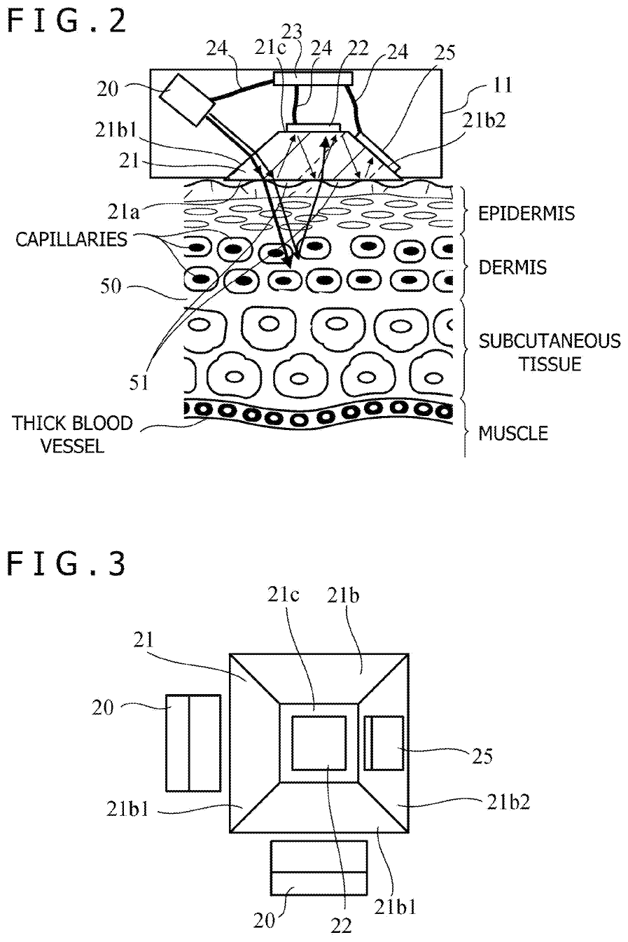

FIG. 2 illustrates the housing 11 in cross section along a plane lying parallel to the sheet of FIG. 1. The housing 11 accommodates the optical system therein as described above. The optical system includes at least one light-emitting element 20, a trapezoidal prism 21, a first light-detecting element 22, and at least one second light-detecting element 25. The housing 11 also houses therein a drive circuit 23 that is connected to the light-emitting element 20, the first light-detecting element 22, and the second light-detecting element 25 by wires 24. The drive circuit 23 and the wires 24 are mounted on a flexible printed circuit board, for example.

The light-emitting element 20 is a semiconductor laser or a light-emitting diode (LED), for example, and emits near-infrared light in a wavelength range of 780 to 2500 nm.

The trapezoidal prism 21 is used to refract the light emitted from the light-emitting element 20 toward the arm 50 and to transmit therethrough light that has been reflected or scattered by a region inside the arm 50. In FIG. 2, the arrows represent paths of the light in the arm 50 and the prism 21.

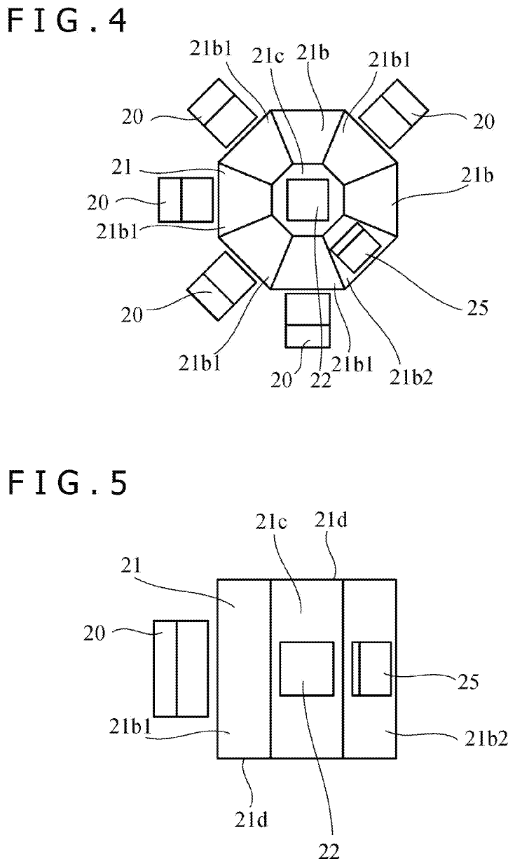

The trapezoidal prism 21 has a trapezoidal, except rectangular, contour as viewed along a certain direction. Specifically, the trapezoidal prism 21 includes two sides parallel to each other and has a certain thickness between the two parallel sides. As illustrated in cross section along a plane in FIG. 2 and viewed along a certain direction perpendicular to the sheet of FIG. 2, the trapezoidal prism 21 has a trapezoidal, except rectangular, contour defined by the two sides and other two sides joining the two sides at their ends. FIG. 3 illustrates, in plan, light-emitting elements 20, the trapezoidal prism 21, the first light-detecting element 22, and the second light-detecting element 25 of the sensor device 10 according to the embodiment. In FIG. 3, the sensor device 10 includes two light-emitting elements 20. FIG. 4 illustrates, in plan, light-emitting elements 20, the trapezoidal prism 21, the first light-detecting element 22, and the second light-detecting element 25 of a sensor device 10 according to a modification of the embodiment. In FIG. 4, the sensor device 10 includes five light-emitting elements 20. FIG. 5 illustrates, in plan, the light-emitting element 20, the trapezoidal prism 21, the first light-detecting element 22, and the second light-detecting element 25 of a sensor device 10 according to another modification of the embodiment. In FIG. 5, the sensor device 10 includes the single light-emitting element 20.

As illustrated in FIGS. 2 and 3, the trapezoidal prism 21 is more accurately shaped as a truncated pyramid. As illustrated in FIG. 3, the two light-emitting elements 20 are disposed in facing relation to two of the four slanted surfaces of the trapezoidal prism 21, and the second light-detecting element 25 is disposed on one of the four slanted surfaces. As illustrated in FIG. 4, the trapezoidal prism 21 according to the modification is shaped as an octagonal truncated pyramid. The five light-emitting elements 20 are disposed in facing relation to five of the eight slanted surfaces of the trapezoidal prism 21, and the second light-detecting element 25 is disposed on one of the eight slanted surfaces. As illustrated in FIG. 5, the trapezoidal prism 21 according to the other modification is shaped as a trapezoidal column. The single light-emitting element 20 is disposed in facing relation to one of the two slanted surfaces of the trapezoidal prism 21, and the second light-detecting element 25 is disposed on the other slanted surface.

As illustrated in FIG. 5, the trapezoidal prism 21 shaped as the trapezoidal column has a first bottom surface 21a, multiple slanted surfaces 21b, and a second bottom surface 21c. The first bottom surface 21a corresponds to the lower bottom of a trapezoid, and the second bottom surface 21c corresponds to the upper bottom of the trapezoid. The second bottom surface 21c lies parallel to the first bottom surface 21a and is smaller in area than the first bottom surface 21a. The terms “bottom surface,” “lower bottom,” and “upper bottom” refer to geometrical terms of a trapezoid and should not be construed as limiting the orientations of the optical system in use.

The slanted surfaces 21b include a first slanted surface 21b1 and a second slanted surface 21b2. The slanted surfaces 21b joins the first bottom surface 21a and the second bottom surface 21c to each other. The trapezoidal prism 21 illustrated in FIG. 5 further has two side surfaces 21d lying parallel to each other.

FIG. 2 illustrates the manner in which the first bottom surface 21a is held in surface contact with the surface of the arm 50 with the degree of close contact that is sufficient for the sensor device 10 to estimate the concentration of the biological component in the arm 50. Although multiple infinitesimal spaces 51 may be present between the first bottom surface 21a and the surface of the arm 50, the first bottom surface 21a is kept in surface contact with the surface of the arm 50 despite those infinitesimal spaces 51.

As illustrated in FIG. 5, the sensor device 10 has the single light-emitting element 20. As illustrated in FIGS. 3 and 4, the respective sensor devices 10 have the multiple light-emitting elements 20. At any rate, each light-emitting element 20 is disposed in facing relation to one slanted surface 21b to apply emitted light to the slanted surface 21b. The slanted surface 21b to which the light emitted from the light-emitting element 20 will hereinafter be referred to as the “first slanted surface 21b1” as described above. The trapezoidal prism 21 refracts the light emitted from the light-emitting element 20 and applied to the first slanted surface 21b1 toward the arm 50.

Another prism, not illustrated, may be interposed between the light-emitting element 20 and the first slanted surface 21b1 to adjust the angle of incidence of the light on the first slanted surface 21b1.

The first light-detecting element 22 is a photodiode, for example, for converting the light it has detected into an electric signal. The first light-detecting element 22 is disposed in facing relation to the second bottom surface 21c of the trapezoidal prism 21. Of the light emitted from the light-emitting element 20, the light, indicated by the thicker arrows in FIG. 2, that has been reflected or scattered by the region inside the arm 50, transmitted through the trapezoidal prism 21, and left the trapezoidal prism 21 from the second bottom surface 21c is detected by the first light-detecting element 22.

The second light-detecting element 25 is also a photodiode, for example, for converting the light it has detected into an electric signal. The second light-detecting element 25 is disposed in facing relation to one of the slanted surfaces 21b of the trapezoidal prism 21. The slanted surface 21b that is faced by the second light-detecting element 25 will hereinafter be referred to as the “second slanted surface 21b2.” The second slanted surface 21b2 is positioned opposite the first slanted surface 21b1 across the second bottom surface 21c. Of the light emitted from the light-emitting element 20, the light that has been reflected from the first bottom surface 21a of the trapezoidal prism 21 into the trapezoidal prism 21, repeatedly reflected within the trapezoidal prism 21, and left the trapezoidal prism 21 from the second slanted surface 21b2 is detected by the second light-detecting element 25. The light reflected within the trapezoidal prism 21 is indicated by thinner arrows in FIG. 2.

As illustrated in FIG. 5, the total number of the light-emitting element 20 and the second light-detecting element 25 is 2 and equal to the number of the slanted surfaces 21b of the trapezoidal prism 21. However, as illustrated in FIGS. 3 and 4, the total number of the light-emitting elements 20 and the second light-detecting element 25 is not equal to the number of the slanted surfaces 21b of the trapezoidal prism 21. In other words, the total number of the first slanted surfaces 21b1 and the second slanted surface 21b2 does not need to be equal to the number of the slanted surfaces 21b.

The drive circuit 23 energizes the light-emitting element 20, the first light-detecting element 22, and the second light-detecting element 25. The drive circuit 23 selectively activates and inactivates the light-emitting element 20, the first light-detecting element 22, and the second light-detecting element 25 in response to commands that are supplied from the controller 18 through a wireless or wired link. Moreover, the drive circuit 23 transmits outputs from the first light-detecting element 22 and the second light-detecting element 25 to the controller 18 through a wireless or wired link.

The controller 18 functions as an estimating section for estimating the concentration of a biological component contained in the human body from the output of the first light-detecting element 22.

As illustrated in FIG. 2, the light emitted from the light-emitting element 20 is refracted by the first slanted surface 21b1 of the trapezoidal prism 21, is further refracted by the first bottom surface 21a and the surface of the arm 50, and travels into the arm 50. Part of the light is absorbed by the dermis in the arm 50 whereas the remainder of the light is reflected back toward the trapezoidal prism 21. The light reflected back toward the trapezoidal prism 21 is refracted by the surface of the arm 50 and the first bottom surface 21a of the trapezoidal prism 21 and travels in the trapezoidal prism 21 before reaching the first light-detecting element 22.

According to the embodiment, the light as it is emitted from the light-emitting element 20 is of such an intensity that the light from the dermis, i.e., mainly the light reflected by the dermis, reaches the first light-detecting element 22. In addition, the direction along which the light emitted from the light-emitting element 20 travels is determined such that the light from the dermis that reaches the first light-detecting element 22 is of a maximum level of intensity. Consequently, the trapezoidal prism 21 is used to determine the direction of travel of the light emitted from the light-emitting element 20.

Since the light reflected from the dermis is used to estimate the concentration of the biological component, the amount of electric power consumed by the light-emitting element 20 is relatively low according to the embodiment.

The dermis contains arterioles, venules, and capillaries. Therefore, the dermis allows glucose to have a high degree of tissue penetration. As glucose is carried by blood in human bodies, it is contained at a high level in the dermis as well as the arteries and the veins. Part of glucose binds to hemoglobin.

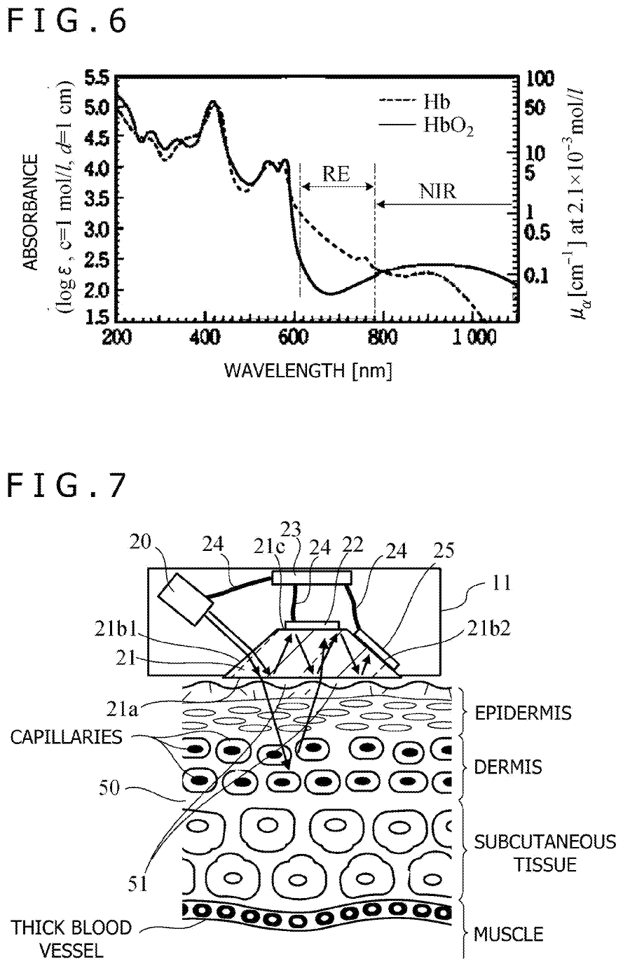

FIG. 6 illustrates optical absorption spectrums of oxygenated hemoglobin (HbO₂) and reduced hemoglobin (Hb). In FIG. 6, NIR represents a near-infrared light wavelength range of 780 to 2500 nm and RE a red light wavelength range of 610 to 780 nm.

As illustrated in FIG. 6, oxygenated hemoglobin absorbs more visible light except red light than near-infrared light, and reduced hemoglobin absorbs more visible light than near-infrared light. Inasmuch as both oxygenated hemoglobin and reduced hemoglobin are less liable to absorb near-infrared light, much of the light from the dermis reaches the first light-detecting element 22 providing that contains capillaries near-infrared light is emitted from the light-emitting element 20.

Consequently, the output of the first light-detecting element 22 is commensurate with the amount of hemoglobin in the dermis. Moreover, since the reflectance of hemoglobin with respect to near-infrared light differs depending on the amount of glucose that is bonded to hemoglobin, the output of the first light-detecting element 22 is also commensurate with the amount of glucose in the dermis. Therefore, the controller 18 is able to estimate the concentration of glucose in the dermis from the output of the first light-detecting element 22.

Glucose is contained at a high level in thick arteries and veins. However, as no thick arteries and veins exist in the dermis, it is unlikely that the output of the first light-detecting element 22 will be affected by a plethysmogram due to blood vessel pulsations. Accordingly, the controller 18 is capable of obtaining an accurate and stable estimate of the concentration of glucose contained in the body of the examinee from the output of the first light-detecting element 22.

In the case where the multiple light-emitting elements 20 are used (see FIGS. 3 and 4), the light-emitting elements 20 emit light in different wavelength ranges. The light-emitting elements 20 may emit such light simultaneously or in a time-division multiplex fashion. The controller 18 is able to obtain an accurate and stable estimate of the concentration of glucose contained in the body of the examinee from the output of the first light-detecting element 22 on the basis of either the light simultaneously emitted from the multiple light-emitting elements 20 or the light emitted from the multiple light-emitting elements 20 in a time-division multiplex fashion.

However, the sensor device 10 may have only one light-emitting element 20 as illustrated in FIG. 5.

As described above, the controller 18 of the sensor device 10 according to the embodiment estimates the concentration of glucose contained in the examinee using the near-infrared light. However, the controller 18 is allowed to estimate the concentration of any of various other biological components in the examinee with the light-emitting element 20 that emits light other than the near-infrared light.

According to the noninvasive optical measurement technology, light reflected or scattered by a region in a human body is measured. According to the embodiment, therefore, it is preferable that of the light emitted from the light-emitting element 20, the light that has been reflected or scattered by the region inside the arm 50 reach as much as possible the first light-detecting element 22. According to the embodiment, the trapezoidal prism 21 refracts the light emitted from the light-emitting element 20 toward the arm 50 and refracts the light reflected or scattered by the region inside the arm 50 toward the first light-detecting element 22.

Even though the trapezoidal prism 21 and the surface of the arm 50 are held in surface contact with each other, however, the infinitesimal spaces 51 may be present between the first bottom surface 21a and the surface of the arm 50 as illustrated in FIG. 2. The refractive index of the trapezoidal prism 21, the refractive index of air, and the refractive index of the surface of the arm 50 are different from each other. Consequently, in the presence of the infinitesimal spaces 51, the light tends to be refracted or reflected in directions other than desired directions by the first bottom surface 21a, i.e., the interface between the trapezoidal prism 21 and air. If the infinitesimal spaces 51 are large, then the light emitted from the light-emitting element 20 may possibly be reflected in its entirety by the first bottom surface 21a and fail to reach the inside of the arm 50.

FIG. 7 illustrates, in cross section, an example of paths of light in the case where the degree of close contact between the surface of the surface of the arm 50 and the trapezoidal prism 21 is low, i.e., the infinitesimal spaces 51 are large. In the example illustrated in FIG. 7, the light emitted from the light-emitting element 20 is significantly reflected by the first bottom surface 21a of the trapezoidal prism 21 and is then repeatedly reflected within the trapezoidal prism 21. As indicated by the different thicknesses of the arrows, which represent the paths of light, in FIGS. 2 and 7, the amount of the light reflected in the trapezoidal prism 21 is larger, i.e., the amount of the light that reaches the inside of the arm 50 is smaller, in the example of FIG. 7. The larger the infinitesimal spaces 51 are, the larger the amount of the light reflected by the first bottom surface 21a or the interface becomes, resulting in a smaller amount of light reflected or refracted by the region in the arm 50 and reaching the first light-detecting element 22 and hence in a lower level of accuracy of the estimate of the concentration of a biological component, e.g., the concentration of glucose, in the human body.

According to the embodiment, the optical system housed in the housing 11 includes the second light-detecting element 25. Of the light emitted from the light-emitting element 20, the light that has been reflected from the first bottom surface 21a of the trapezoidal prism 21 into the trapezoidal prism 21 and left the trapezoidal prism 21 from the second slanted surface 21b2 is detected by the second light-detecting element 25. The second light-detecting element 25 is provided to monitor the intensity of the light reflected by the first bottom surface 21a. The controller 18 determines whether the degree of close contact between the surface of the surface of the arm 50 and the trapezoidal prism 21 is sufficient for the sensor device 10 to estimate the concentration of the biological component in the arm 50 or not on the basis of the monitored intensity of the light.

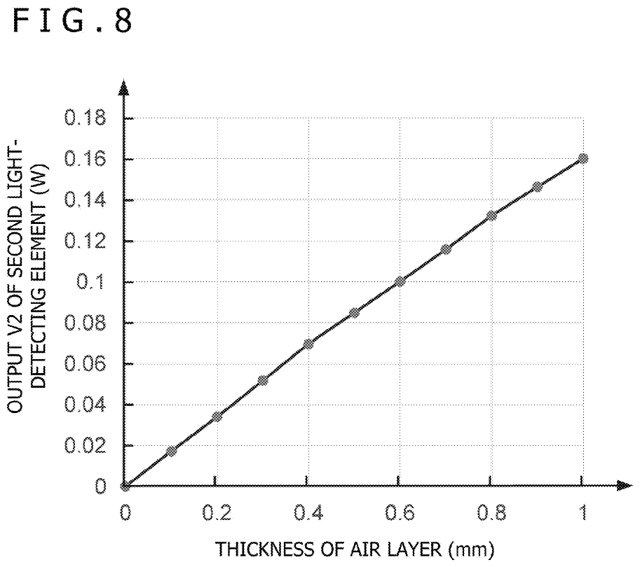

FIG. 8 is a graph illustrating a relation between a thickness of an air layer between the surface of the arm 50 and the trapezoidal prism 21 and the magnitude of the output V2 from the second light-detecting element 25. The graph was obtained from a simulation on the assumption that the air layer has a uniform thickness. It is seen from FIG. 8 that the thicker the air layer is, the larger the output V2 from the second light-detecting element 25 becomes. Although the actual infinitesimal spaces 51 do not have a uniform thickness, it will be understood from a study of FIG. 8 that the larger the infinitesimal spaces 51 are, the stronger the output V2 from the second light-detecting element 25 is.

Therefore, by monitoring the output V2 from the second light-detecting element 25, it is possible to determine whether the first light-detecting element 22 is detecting an amount of light large enough to estimate the concentration of the biological component or not. According to the embodiment, the controller 18 functions as a determining section for determining whether the estimating section can estimate the concentration of the biological component or not on the basis of the output V2 from the second light-detecting element 25.

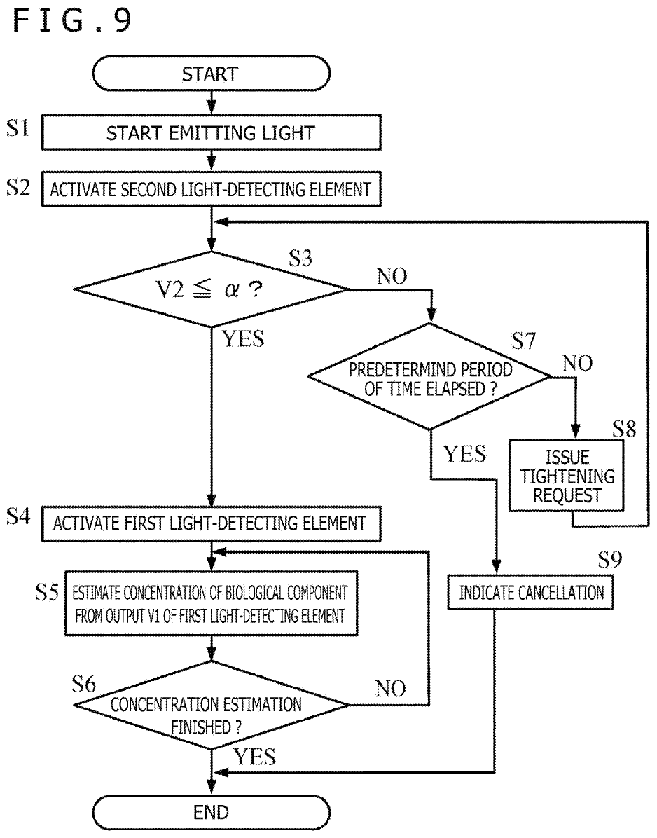

An example of operation of the sensor device 10, i.e., a process of using the sensor device 10, will be described in detail below with reference to a flowchart of FIG. 9.

The sensor device 10 starts to operate when the start button 17a (see FIG. 1) of the switch unit 17 is pressed.

In step S1, the controller 18 supplies a drive signal to the light-emitting element 20 to enable the light-emitting element 20 to start emitting light. In step S1, the controller 18 may supply a drive signal to the single light-emitting element 20 positioned opposite the second light-detecting element 25 across the second bottom surface 21c of the trapezoidal prism 21 or may supply drive signals to the multiple light-emitting elements 20.

In step S2, the controller 18 supplies a drive signal to the second light-detecting element 25, i.e., activates the second light-detecting element 25, to start its photoelectric conversion process. The controller 18 also starts to record the output V2 from the second light-detecting element 25.

In step S3, the controller 18 determines whether or not the magnitude of the output V2 from the second light-detecting element 25 is equal to or smaller than a threshold value α, i.e., a first threshold value. The threshold value α used in step S3 represents a fixed value. The threshold value α is stored in the storage device 19.

If the decision from step S3 is affirmative, then the processing goes to step S4. In step S4, the controller 18 supplies a drive signal to the first light-detecting element 22, i.e., activates the first light-detecting element 22, to start its photoelectric conversion process. The controller 18 also starts to record the output V1 from the first light-detecting element 22.

When the decision from step S3 is affirmative, the output V2 from the second light-detecting element 25 is small, indicating that the infinitesimal spaces 51 that are present between the first bottom surface 21a of the trapezoidal prism 21 and the surface of the arm 50 are small, i.e., the degree of close contact between the surface of the arm 50 and the trapezoidal prism 21 is high. In this case, it is considered that the amount of light that is sufficient to estimate the concentration of the biological component has reached the first light-detecting element 22.

While the degree of close contact between the surface of the arm 50 and the trapezoidal prism 21 is being kept thus adequate, the controller 18 estimates, in step S5, the concentration of the biological component, e.g., the concentration of glucose, included in the human body from the output V1 of the first light-detecting element 22.

Providing the sensor device 10 includes the multiple light-emitting elements 20 and one of them has been energized prior to step S5, the controller 18 may in step S5 energize the other light-emitting element or elements 20 and estimate the concentration of the biological component included in the human body from the output V1 of the first light-detecting element 22 based on the light coming from the multiple light-emitting elements 20. Alternatively, the controller 18 may not energize the other light-emitting element or elements 20 and estimate the concentration of the biological component included in the human body from the output V1 of the first light-detecting element 22 based on the light coming from the single light-emitting element 20.

In step S6, the controller 18 determines whether the concentration estimation has been finished or not. If the concentration estimation has not been finished, i.e., the decision from step S6 is negative, then the processing goes back to step S5.

If the concentration estimation has been finished, i.e., the decision from step S6 is affirmative, then the processing comes to an end.

If the decision from step S3 is negative, then the processing goes to step S7. When the decision from step S3 is negative, the output V2 from the second light-detecting element 25 is large, indicating that the infinitesimal spaces 51 that are present between the first bottom surface 21a of the trapezoidal prism 21 and the surface of the arm 50 are large, i.e., the degree of close contact between the surface of the arm 50 and the trapezoidal prism 21 is low. In this case, it is considered that the amount of light that is sufficient to estimate the concentration of the biological component has not reached the first light-detecting element 22.

In step S7, the controller 18 determines whether a predetermined period of time has elapsed from an initial event of detection that the magnitude of the output V2 of the second light-detecting element 25 is larger than the threshold value α or not. If the decision from step S7 is negative, then the processing goes to step S8. In step S8, the controller 18 supplies a control signal to the indicating device 18A, enabling the indicating device 18A to issue a request, i.e., a tightening request, for prompting the user, i.e., the examinee or the medical attendant, to press the trapezoidal prism 21 strongly against the arm 50. If the indicating device 18A is a display device, then the tightening request represents a message of letters. In the indicating device 18A is a speaker, then the tightening request represents speech sounds or beeps. The tightening request indicates that the tightening force applied to press the trapezoidal prism 21 against the arm 50 is not strong enough.

In response to the tightening request, the user can operate the buckle 13c of the belt 13 to press the trapezoidal prism 21 more strongly against the arm 50. After step S8, the processing goes back to step S3.

Steps S3, S7, and S8 provide a control loop for giving the user an opportunity to press the trapezoidal prism 21 more strongly against the arm 50 to increase the degree of close contact between the surface of the surface of the arm 50 and the trapezoidal prism 21.

If the decision from step S7 is affirmative, then the processing proceeds to step S9. In other words, if the predetermined period of time has elapsed from the initial event of detection that the magnitude of the output V2 of the second light-detecting element 25 is larger than the threshold value α and thereafter the magnitude of the output V2 of the second light-detecting element 25 nevertheless remains larger than the threshold value α, then the processing proceeds to step S9. In step S9, the controller 18 controls the indicating device 18A to give the user a canceling indication that the estimation of the concentration of the biological component is to be canceled, after which the processing comes to an end, i.e., the estimating section stops estimating the concentration of the biological component. If the indicating device 18A is a display device, then the canceling indication may represent a message of letters. In the indicating device 18A is a speaker, then the canceling indication may represent speech sounds or beeps.

As described above, the controller 18 decides that it can estimate the concentration of the biological component if the magnitude of the output V2 of the second light-detecting element 25 is equal to or smaller than the threshold value α, and decides that it is not allowed to estimate the concentration of the biological component if the magnitude of the output V2 of the second light-detecting element 25 is larger than the threshold value α. If the magnitude of the output V2 of the second light-detecting element 25 is larger than the threshold value α, then the degree of close contact between the surface of the arm 50 and the trapezoidal prism 21 is low and the amount of light detected by the first light-detecting element 22 is small and not enough. On the other hand, if the magnitude of the output V2 of the second light-detecting element 25 is equal to or smaller than the threshold value α, then the degree of close contact between the surface of the arm 50 and the trapezoidal prism 21 is high and the amount of light detected by the first light-detecting element 22 is large and enough.

The controller 18 thus determines whether it can estimate the concentration of the biological component or not on the basis of the output V2 from the second light-detecting element 25. Therefore, if the degree of close contact between the surface of the arm 50 and the trapezoidal prism 21 is insufficient, then the controller 18 does not estimate the concentration of the biological component, and if the degree of close contact between the surface of the arm 50 and the trapezoidal prism 21 is sufficient, then the controller 18 estimates the concentration of the biological component.

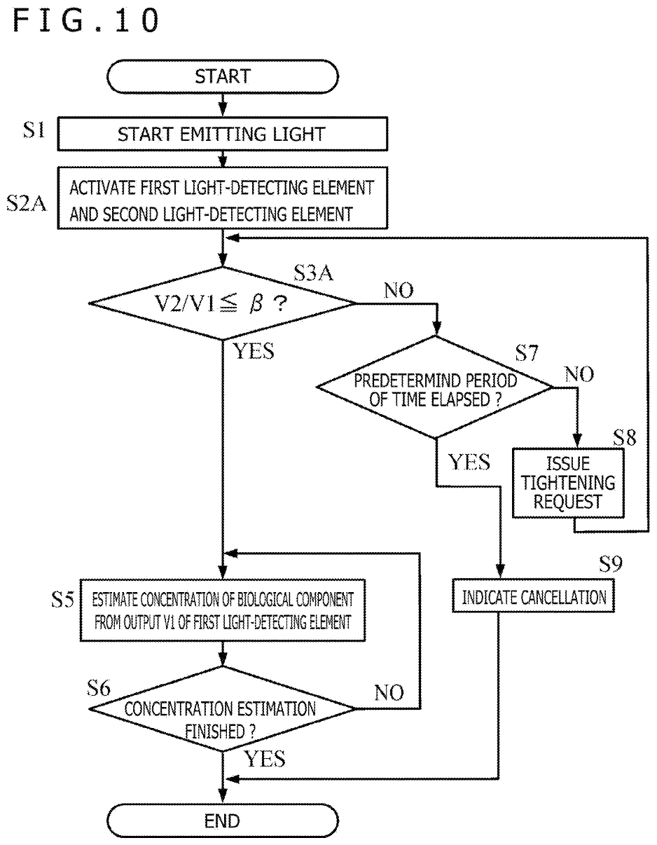

FIG. 10 is a flowchart illustrating another example of operation of the sensor device 10, i.e., another process of using the sensor device 10. The flowchart of FIG. 10 shares certain steps with the flowchart of FIG. 9. The example of operation of the sensor device 10 illustrated in FIG. 10 will be described below primarily with respect to steps that are different from the steps of the example of operation of the sensor device 10 illustrated in FIG. 9.

According to the example of operation illustrated in FIG. 10, the controller 18 carries out step S2A instead of step S2 after step S1. In step S2A, the controller 18 supplies drive signals to the first light-detecting element 22 and the second light-detecting element 25, i.e., activates the first light-detecting element 22 and the second light-detecting element 25, to start their photoelectric conversion processes. The controller 18 also starts to record the output V1 from the first light-detecting element 22 and the output V2 from the second light-detecting element 25.

According to the example of operation of FIG. 10, the controller 18 carries out step S3A instead of step S3 in FIG. 9 after step S2A. In step S3A, the controller 18 determines whether or not the ratio V2/V1 of the output V2 of the second light-detecting element 25 to the output V1 of the first light-detecting element 22 is equal to or smaller than a threshold value β, i.e., a second threshold value. The threshold value β used in step S3A represents a fixed value. The threshold value β is stored in the storage device 19.

If the decision from step S3A is affirmative, then the processing goes to step S5. Since the first light-detecting element 22 has been activated in step S2A, step S4 in FIG. 9 is not necessary in the flowchart of FIG. 10.

When the decision from step S3A is affirmative, the output V2 of the second light-detecting element 25 is small and the output V1 of the first light-detecting element 22 is large. At this time, the infinitesimal spaces 51 that are present between the first bottom surface 21a of the trapezoidal prism 21 and the surface of the arm 50 are small, i.e., the degree of close contact between the surface of the arm 50 and the trapezoidal prism 21 is high. In this case, it is considered that the amount of light that is sufficient to estimate the concentration of the biological component has reached the first light-detecting element 22.

While the degree of close contact between the surface of the arm 50 and the trapezoidal prism 21 is being kept thus adequate, the controller 18 estimates, in step S5, the concentration of the biological component, e.g., the concentration of glucose, included in the human body from the output V1 of the first light-detecting element 22.

Providing the sensor device 10 includes the multiple light-emitting elements 20 and one of them has been energized prior to step S5, the controller 18 may in step S5 energize the other light-emitting element or elements 20 and estimate the concentration of the biological component included in the human body from the output V1 of the first light-detecting element 22 based on the light coming from the multiple light-emitting elements 20. Alternatively, the controller may not energize the other light-emitting element or elements and estimate the concentration of the biological component included in the human body from the output V1 of the first light-detecting element 22 based on the light coming from the single light-emitting element 20.

If the decision from step S3A is negative, then the processing goes to step S7. When the decision from step S3A is negative, the output V2 from the second light-detecting element 25 is large and the output V1 from the first light-detecting element 22 is small, indicating that the infinitesimal spaces 51 that are present between the first bottom surface 21a of the trapezoidal prism 21 and the surface of the arm 50 are large, i.e., the degree of close contact between the surface of the arm 50 and the trapezoidal prism 21 is low. In this case, it is considered that the amount of light that is sufficient to estimate the concentration of the biological component has not reached the first light-detecting element 22.

In step S7, the controller 18 determines whether a predetermined period of time has elapsed from an initial event of detection that the ratio V2/V1 is larger than the threshold value β. If the decision from step S7 is negative, then the processing goes to step S8. In step S8, the controller 18 supplies a control signal to the indicating device 18A, enabling the indicating device 18A to issue a request, i.e., a tightening request, for prompting the user to press the trapezoidal prism 21 strongly against the arm 50. If the indicating device 18A is a display device, then the tightening request may represent a message of letters. In the indicating device 18A is a speaker, then the tightening request may represent speech sounds or beeps. The tightening request indicates that the tightening force applied to press the trapezoidal prism 21 against the arm 50 is not strong enough.

In response to the tightening request, the user can operate the buckle 13c of the belt 13 to press the trapezoidal prism 21 more strongly against the arm 50. After step S8, the processing goes back to step S3A.

Steps S3A, S7, and S8 provide a control loop for giving the user (the examinee or the medical attendant) an opportunity to press the trapezoidal prism 21 more strongly against the arm 50 to increase the degree of close contact between the surface of the surface of the arm 50 and the trapezoidal prism 21.

If the decision from step S7 is affirmative, then the processing proceeds to step S9. Consequently, if the predetermined period of time has elapsed from the initial event of detection that the magnitude of the output V2 of the second light-detecting element 25 is larger than the threshold value β and thereafter the magnitude of the output V2 of the second light-detecting element 25 nevertheless remains larger than the threshold value β, then the processing proceeds to step S9. In step S9, the controller 18 controls the indicating device 18A to give the user a canceling indication that the estimation of the concentration of the biological component is to be canceled, after which the processing comes to an end, i.e., the estimating section stops estimating the concentration of the biological component. If the indicating device 18A is a display device, then the canceling indication represents a message of letters. In the indicating device 18A is a speaker, then the canceling indication represents speech sounds or beeps.

As described above, the controller 18 decides that it can estimate the concentration of the biological component if the ratio V2/V1 of the output V2 of the second light-detecting element 25 to the output V1 of the first light-detecting element 22 is equal to or smaller than the threshold value β, and decides that it is not allowed to estimate the concentration of the biological component if the ratio V2/V1 is larger than the threshold value β. If the ratio V2/V1 is larger than the threshold value β, then the degree of close contact between the surface of the arm 50 and the trapezoidal prism 21 is low and the amount of light detected by the first light-detecting element 22 is small and not enough. On the other hand, if the ratio V2/V1 is equal to or smaller than the threshold value β, then the degree of close contact between the surface of the arm 50 and the trapezoidal prism 21 is high and the amount of light detected by the first light-detecting element 22 is large and enough.

The controller 18 thus determines whether it can estimate the concentration of the biological component or not on the basis of the output V2 from the second light-detecting element 25, particularly the ratio V2/V1. Therefore, if the degree of close contact between the surface of the arm 50 and the trapezoidal prism 21 is insufficient, then the controller 18 does not estimate the concentration of the biological component, and if the degree of close contact between the surface of the arm 50 and the trapezoidal prism 21 is sufficient, then the controller 18 estimates the concentration of the biological component.

Now, a modification of the embodiment for automatically adequately controlling the degree of close contact between the surface of the arm 50 and the trapezoidal prism 21 will be described below.

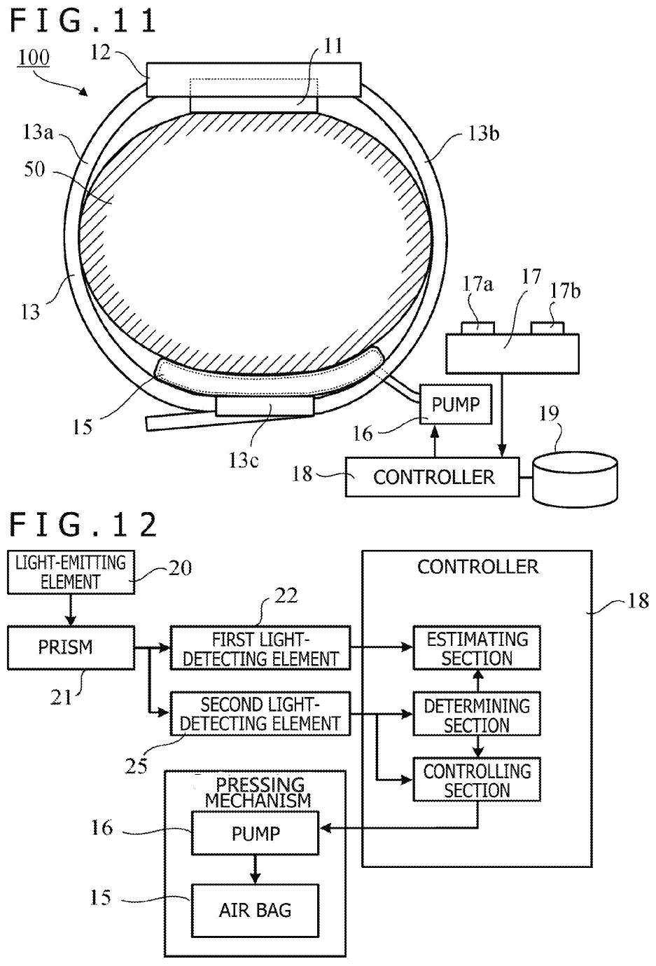

FIG. 11 illustrates, in front elevation, a sensor device 100 according to the modification of the embodiment. The sensor device 100 is of basically the same structure as the sensor device 10 and additionally has an air bag 15 and a pump 16. Though the sensor device 100 is free of the indicating device 18A of the sensor device 10, the sensor device 100 may have the indicating device 18A.

The air bag 15 is sandwiched between the belt 13 and the human body, e.g., the arm 50. The pump 16 delivers air into the air bag 15. The air bag 15 and the pump 16 jointly make up a pressing mechanism for pressing the optical system in the housing 11, particularly the trapezoidal prism 21, against the human body.

Specifically, when the pump 16 is actuated, it delivers air into the air bag 15, expanding the air bag 15. When the air bag 15 is expanded, it exerts a tensile force to the belt 13, causing the support 12 to press the housing 11 toward the arm 50. Therefore, the optical system in the housing 11, particularly the trapezoidal prism 21, is pressed against the arm 50.

The switch unit 17 has the start button 17a and a stop button 17b. When the examinee or the medical attendant presses the start button 17a, the switch unit 17 supplies the controller 18 with a start command, enabling the sensor device 10 to start measuring the concentration of the biological component of the examinee. When the examinee or the medical attendant presses the stop button 17b, the switch unit 17 supplies the controller 18 with a stop command, causing the sensor device 10 to stop measuring the concentration of the biological component of the examinee.

The controller 18 is connected to the switch unit 17 through a wireless or wired link for receiving the start command and the stop command from the switch unit 17. The controller 18 is also connected to the pump 16 through a wireless or wired link for supplying a drive signal to the pump 16.

The controller 18 functions as a controlling section for controlling the pump 16. Specifically, the controller 18 controls the pump 16 on the basis of the output V1 of the first light-detecting element 22 to enable the air bag 15 to automatically increase the pressing force applied from the trapezoidal prism 21 to the arm 50. As a result, the controller 18 increases to a certain extent the degree of close contact between the surface of the arm 50 and the trapezoidal prism 21.

Moreover, the controller 18 controls the pump 16 on the basis of the output V2 of the second light-detecting element 25 to enable the air bag 15 to automatically adequately adjust the pressing force applied from the trapezoidal prism 21 to the arm 50. As a result, the controller 18 adequately adjusts the degree of close contact between the surface of the arm 50 and the trapezoidal prism 21.

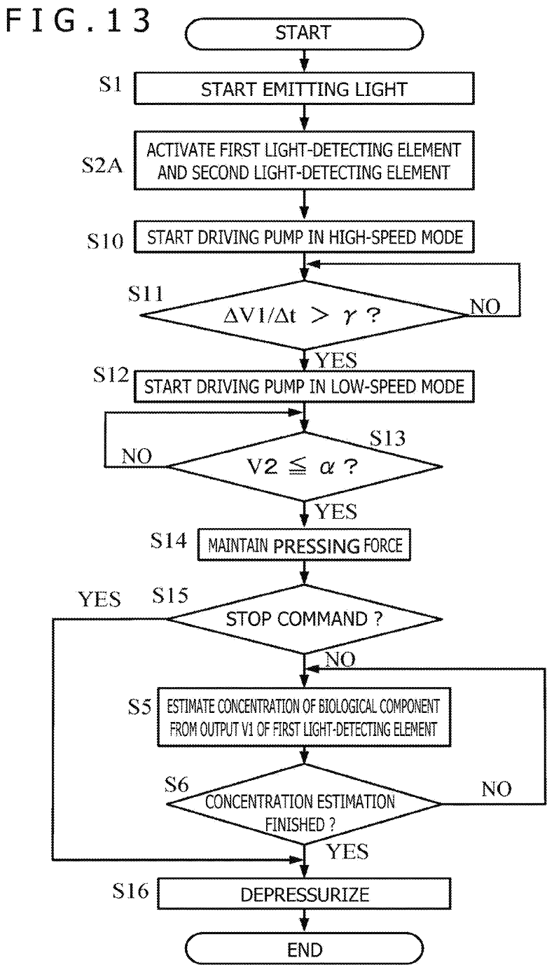

The sensor device 100 has a control system illustrated in FIG. 12.

The sensor device 100 may have another pressing mechanism instead of the air bag 15 and the pump 16. The other pressing mechanism may include a hydraulic mechanism, a linear actuator, or a cam mechanism, for example.

An example of operation of the sensor device 100 illustrated in FIG. 11, i.e., a process of using the sensor device 100, will be described in detail below with reference to a flowchart of FIG. 13.

The sensor device 100 starts to operate when the start button 17a (see FIG. 11) of the switch unit 17 is pressed.

The flowchart of FIG. 13 shares certain steps with the flowchart of FIGS. 9 and 10. The example of operation of the sensor device 100 illustrated in FIG. 13 will be described below primarily with respect to steps that are different from the steps of the examples of operation of the sensor device 10 illustrated in FIGS. 9 and 10.

In step S1, the controller 18 supplies a drive signal to the light-emitting element 20 to enable the light-emitting element 20 to start emitting light. In step S2A, the controller 18 activates the first light-detecting element 22 and the second light-detecting element 25. Then, the processing proceeds to step S10.

In step S10, the controller 18 supplies a drive signal to the pump 16, starting to actuate the pump 16. The actuated pump 16 starts expanding the air bag 15. In step S10, specifically, the controller 18 starts driving the pump 16 in a high-speed mode, i.e., a first mode. The high-speed mode refers to a mode of driving the pump 16 at a high speed until the trapezoidal prism 21 can press the arm 50 with a certain pressing force.

In step S11, the controller 18 determines whether a change ΔV1/Δt in the output V1 of the first light-detecting element 22 per unit time Δt is larger than a threshold value γ, i.e., a third threshold value, or not. If the decision from step S11 is negative, then the processing loops back to step S11.

In other words, the controller 18 waits until the change ΔV1/Δt in the output V1 of the first light-detecting element 22 per unit time Δt reaches the threshold value γ, i.e., becomes equal to or larger than the threshold value γ. Stated otherwise, the pump 16 keeps being driven in the high-speed mode until the decision from step S11 becomes affirmative. Since the controller 18 continuously drives the pump 16 in the high-speed mode during an initial stage of its control over the pump 16, the air bag 15 is expanded quickly.

If the decision from step S11 is affirmative, then the processing goes to step S12. In step S12, the controller 18 starts driving the pump 16 in a low-speed mode, i.e., a second mode. In the low-speed mode, the pump 16 is driven to supply air at a lower rate than the rate at which the pump 16 supplies air in the high-speed mode, thereby reducing the rate at which the air bag 15 is expanded. The low-speed mode refers to a mode of driving the pump 16 at a low speed after the trapezoidal prism 21 has pressed the arm 50 with the certain pressing force.

After the air bag 15 has started to expand in step S10, while the amount of inflating air in the air bag 15 is small in the initial stage, i.e., during the high-speed mode, the air bag 15 makes no or almost no contribution to the pressurization of the trapezoidal prism 21. Therefore, during the initial stage, the distance between the trapezoidal prism 21 and the dermis (see FIGS. 2 and 7) remains so large that the output V1 of the first light-detecting element 22 does not essentially increase.

After the amount of inflating air in the air bag 15 has become large to a certain extent at the end of the initial stage, the air bag 15 is expanded enough to press the housing 11 toward the arm 50. Therefore, the trapezoidal prism 21 is pressed against the arm 50, resulting in an increase in the output V1 of the first light-detecting element 22.

During the initial stage, the controller 18 drives the pump 16 in the high-speed mode to expand the air bag 15 at a high expansion rate. After the initial stage, the controller 18 drives the pump 16 in the low-speed mode to expand the air bag 15 at a low expansion rate. In the example of operation illustrated in FIG. 13, the stage until the change ΔV1/Δt in the output V1 of the first light-detecting element 22 per unit time Δt reaches the threshold value γ, i.e., until the decision from step S11 becomes affirmative, represents the initial stage. The threshold value γ is stored in the storage device 19. The threshold value γ represents a fixed value.

After step S12, the controller 18 determines whether or not the magnitude of the output V2 from the second light-detecting element 25 is equal to or smaller than a threshold value, i.e., a first threshold value, in step S13. The threshold value α used in step S13 represents a fixed value. The threshold value α is stored in the storage device 19.

If the decision from step S13 is negative, then the processing loops back to step S13. Therefore, the controller 18 continuously drives the pump 16 in the low-speed mode. In other words, until the magnitude of the output V2 from the second light-detecting element 25 becomes equal to or smaller than the threshold value α, the controller 18 controls the pump 16 to expand the air bag 15 in the low-speed mode, thereby increasing the pressing force applied from the trapezoidal prism 21 to the arm 50. The infinitesimal spaces 51 between the first bottom surface 21a of the trapezoidal prism 21 and the surface of the arm 50 are thus reduced, i.e., the degree of close contact between the surface of the arm 50 and the trapezoidal prism 21 is increased.

On the other hand, if the decision from step S13 is affirmative, i.e., the magnitude of the output V2 from the second light-detecting element 25 is equal to or smaller than the threshold value α, then the processing goes to step S14. In step S14, the controller 18 gives a command to the pump 16 to stop increasing the pressing force. Stated otherwise, the pressing force applied from the trapezoidal prism 21 to the arm 50 is maintained, thereby maintaining the degree of close contact between the surface of the arm 50 and the trapezoidal prism 21.

In step S15 after step S14, the controller 18 determines whether it has received a stop command from the switch unit 17 or not. If the decision from step S15 is affirmative, then the processing goes to step S16. In step S16, the controller 18 gives a command to the pump 16 to depressurize the air bag 15 in order to reduce the pressing force applied from the trapezoidal prism 21 to the arm 50. In response to the command, the pump 16 discharges air from the air bag 15.

Thereafter, the processing comes to an end. If the examinee feels pain from being tightened by the belt 13 or pressed by the trapezoidal prism 21, for example, the examinee or the medical attendant presses the stop button 17b, whereupon the switch unit 17 supplies a stop command to the controller 18. Now, the decision from step S15 becomes affirmative, stopping the measurement of the concentration of the biological component.

If the decision from step S15 is negative, the processing goes to step S5. While the degree of close contact between the surface of the arm 50 and the trapezoidal prism 21 is being kept adequate in step S14, the controller 18 estimates, in step S5, the concentration of the biological component, e.g., the concentration of glucose, included in the human body from the output V1 of the first light-detecting element 22.

Providing the sensor device 10 includes the multiple light-emitting elements 20 and one of them has been energized prior to step S5, the controller 18 may in step S5 energize the other light-emitting element or elements 20 and estimate the concentration of the biological component included in the human body from the output V1 of the first light-detecting element 22 based on the light coming from the multiple light-emitting elements 20. Alternatively, the controller 18 may not energize the other light-emitting element or elements 20 and estimate the concentration of the biological component included in the human body from the output V1 of the first light-detecting element 22 based on the light coming from the single light-emitting element 20.

In step S6, the controller 18 determines whether the concentration estimation has been finished or not. If the concentration estimation has not been finished, i.e., the decision from step S6 is negative, then the processing goes back to step S5.

If the concentration estimation has been finished, i.e., the decision from step S6 is affirmative, then the processing proceeds to step S16.

In step S16, the controller 18 gives a command to the pump 16 to depressurize the air bag 15 in order to reduce the pressing force applied from the trapezoidal prism 21 to the arm 50. In response to the command, the pump 16 discharges air from the air bag 15.

After step S16, the processing comes to an end.

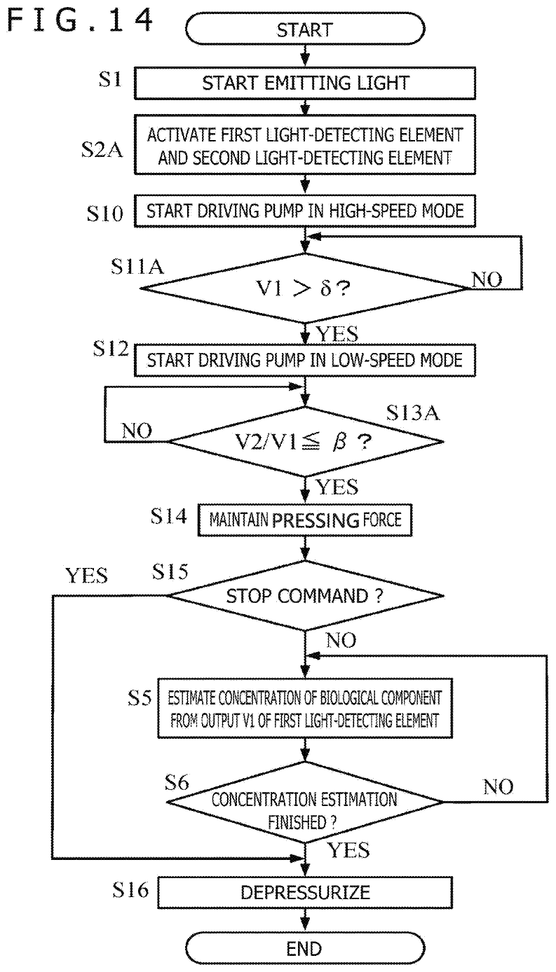

FIG. 14 illustrates a flowchart of another example of operation of the sensor device 100. The flowchart of FIG. 14 shares certain steps with the flowchart of FIG. 13. The example of operation of the sensor device 100 illustrated in FIG. 14 will be described below primarily with respect to steps that are different from the steps of the example of operation of the sensor device 100 illustrated in FIG. 13.

According to the example of operation illustrated in FIG. 14, step S10 is followed by step S11A instead of step S11. In step S11A, the controller 18 determines whether the magnitude of the output V1 of the first light-detecting element 22 is larger than a threshold value δ, i.e., a fourth threshold value, or not. If the decision from step S11A is negative, then the processing loops back to step S11A.

In other words, the controller 18 waits until the magnitude of the output V1 of the first light-detecting element 22 reaches the threshold value δ, i.e., becomes equal to or larger than the threshold value δ. Stated otherwise, the pump 16 keeps being driven in the high-speed mode until the decision from step S11A becomes affirmative. Since the controller 18 continuously drives the pump 16 in the high-speed mode during the initial stage of its control over the pump 16, the air bag 15 is expanded quickly.

If the decision from step S11A is affirmative, then the processing goes to step S12. In step S12, the controller 18 starts driving the pump 16 in the low-speed mode. In the low-speed mode, the pump 16 is driven to supply air at a lower rate than the rate at which the pump 16 supplies air in the high-speed mode, thereby reducing the rate at which the air bag 15 is expanded.

During the initial stage, the controller 18 drives the pump 16 in the high-speed mode to expand the air bag 15 at a high expansion rate. After the initial stage, the controller 18 drives the pump 16 in the low-speed mode to expand the air bag 15 at a low expansion rate. In the example of operation illustrated in FIG. 14, the stage until the magnitude of the output V1 of the first light-detecting element 22 reaches the threshold value δ, i.e., until the decision from step S11A becomes affirmative, represents the initial stage. The threshold value δ is stored in the storage device 19. The threshold value d represents a fixed value.

According to the example of operation illustrated in FIG. 14, step S12 is followed by step S13A instead of step S13. In step S13A, the controller 18 determines whether or not the ratio V2/V1 of the output V2 of the second light-detecting element 25 to the output V1 of the first light-detecting element 22 is equal to or smaller than a threshold value β, i.e., a second threshold value. The threshold value β used in step S13A represents a fixed value. The threshold value β is stored in the storage device 19.

If the decision from step S13A is negative, then the processing loops back to step S13A. Therefore, the controller 18 continuously drives the pump 16 in the low-speed mode. In other words, until the ratio V2/V1 becomes equal to or smaller than the threshold value β, the controller 18 controls the pump 16 to expand the air bag 15 in the low-speed mode, thereby increasing the pressing force applied from the trapezoidal prism 21 to the arm 50. The infinitesimal spaces 51 between the first bottom surface 21a of the trapezoidal prism 21 and the surface of the arm 50 are thus reduced, i.e., the degree of close contact between the surface of the arm 50 and the trapezoidal prism 21 is increased.

On the other hand, if the decision from step S13A is affirmative, i.e., the ratio V2/V1 is equal to or smaller than the threshold value β, then the processing goes to step S14. In step S14, the controller 18 gives a command to the pump 16 to stop increasing the pressing force. Stated otherwise, the pressing force applied from the trapezoidal prism 21 to the arm 50 is maintained, thereby maintaining the degree of close contact between the surface of the arm 50 and the trapezoidal prism 21.

Thereafter, unless the controller 18 decides that it has received a stop command in step S15, the processing goes to step S5. While the degree of close contact between the surface of the arm 50 and the trapezoidal prism 21 is being kept adequate in step S14, the controller 18 estimates, in step S5, the concentration of the biological component, e.g., the concentration of glucose, included in the human body from the output V1 of the first light-detecting element 22.

According to the examples of operation illustrated in FIGS. 13 and 14, the controller 18 automatically adequately adjusts the degree of close contact between the surface of the arm 50 and the trapezoidal prism 21. While the degree of close contact between the surface of the arm 50 and the trapezoidal prism 21 is being kept adequate, the controller 18 estimates, in step S5, the concentration of the biological component, e.g., the concentration of glucose, included in the human body from the output V1 of the first light-detecting element 22.

In the example of operation illustrated in FIG. 13, step S11A may be carried out instead of step S11. In the example of operation illustrated in FIG. 14, step S11 may be carried out instead of step S11A.

Alternatively, step S11 illustrated in FIG. 13 or step S11A illustrated in FIG. 14 may not be carried out and the controller 18 may drive the pressing mechanism in the high-speed mode for a predetermined period of time representing the initial stage, and then, after elapse of a predetermined period of time, may start the low-speed mode in step S12.

According to the embodiment, the second light-detecting element 25 is disposed in facing relation to the second slanted surface 21b2 of the trapezoidal prism 21 that is positioned opposite the first slanted surface 21b1 across the second bottom surface 21c. Consequently, the first light-detecting element 22 that is disposed in facing relation to the second bottom surface 21c of the trapezoidal prism 21 may have a large detection area without being adversely affected by the second light-detecting element 25 so as to be able to detect much light reflected or scattered by the region in the arm 50. Furthermore, the second light-detecting element 25 that faces the second slanted surface 21b2 is able to detect much light reflected into the trapezoidal prism 21 by the first bottom surface 21a of the trapezoidal prism 21.

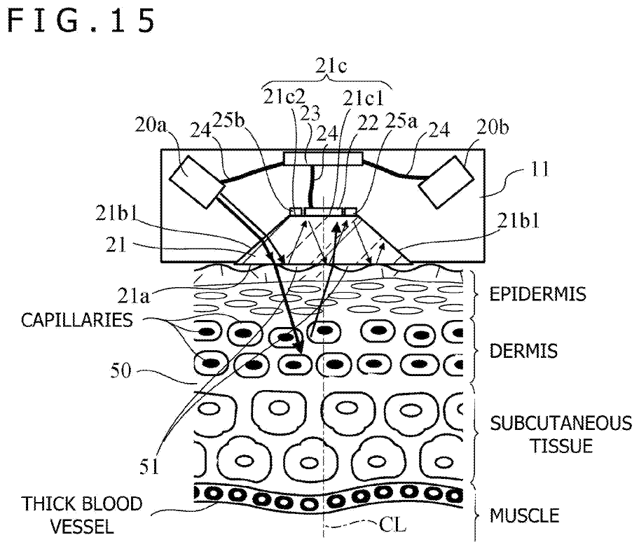

The second light-detecting element may be disposed so as to detect the light emitted from the second bottom surface 21c of the trapezoidal prism 21. According to a modification illustrated in FIG. 15, two second light-detecting elements 25a and 25b are disposed in facing relation to the second bottom surface 21c for detecting the light emitted from the second bottom surface 21c.

The second bottom surface 21c of the trapezoidal prism 21 has a central area 21c1 and a peripheral area 21c2 disposed in the periphery of the central area 21c1. The first light-detecting element 22 is disposed in facing relation to the central area 21c1. The second light-detecting elements 25a and 25b are disposed in facing relation to the peripheral area 21c2.

According to the modification illustrated in FIG. 15, there are two light-emitting elements 20a and 20b in use. The second light-detecting element 25a is disposed opposite the light-emitting element 20a across the first light-detecting element 22 for detecting the light that comes from the light-emitting element 20a and is reflected in the trapezoidal prism 21. The arrows in FIG. 15 indicate the light coming from the light-emitting element 20a. The second light-detecting element 25b is disposed opposite the light-emitting element 20b across the first light-detecting element 22 for detecting the light that comes from the light-emitting element 20b and is reflected in the trapezoidal prism 21. The light that comes from the light-emitting element 20b is not illustrated but is understood as being in line symmetry with the arrows in FIG. 15 with respect to a central line CL of the first light-detecting element 22.

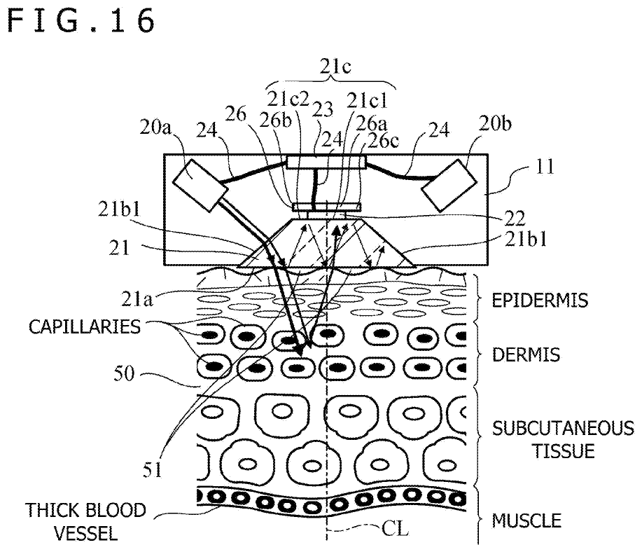

According to another modification illustrated in FIG. 16, a second light-detecting element 26 is placed in overlapping relation to the first light-detecting element 22 in facing relation to the second bottom surface 21c for detecting the light emitted from the second bottom surface 21c.

The first light-detecting element 22 is disposed in facing relation to the central area 21c1.

The second light-detecting element 26 has an area larger than the first light-detecting element 22 and has an overlapping portion 26a overlapping the first light-detecting element 22 and protrusive portions 26b and 26c protruding from the first light-detecting element 22. The protrusive portions 26b and 26c are disposed in facing relation to the peripheral area 21c2.

According to the modification illustrated in FIG. 16, the two light-emitting elements 20a and 20b are employed. The protrusive portion 26c of the second light-detecting element 26 is disposed opposite the light-emitting element 20a across the first light-detecting element 22 for detecting the light that comes from the light-emitting element 20a and is reflected in the trapezoidal prism 21. The arrows in FIG. 16 indicate the light coming from the light-emitting element 20a. The protrusive portion 26b of the second light-detecting element 26 is disposed opposite the light-emitting element 20b across the first light-detecting element 22 for detecting the light that comes from the light-emitting element 20b and is reflected in the trapezoidal prism 21. The light that comes from the light-emitting element 20b is not illustrated but is understood as being in line symmetry with the arrows in FIG. 16 with respect to the central line CL of the first light-detecting element 22.

According to the modifications illustrated in FIGS. 15 and 16, the second light-detecting elements 25a and 25b and the protrusive portions 26b and 26c of the second light-detecting element 26 detect the light emitted from the peripheral area 21c2 of the second bottom surface 21c. Therefore, the area of the first light-detecting element 22 that detects the light emitted from the central area 21c1 of the second bottom surface 21c is limited.

However, since the second light-detecting element does not detect the light emitted from the slanted surfaces 21b, the light-emitting elements 20 may be disposed in facing relation to the slanted surfaces 21b. Accordingly, the number of the light-emitting elements 20 can be increased.

According to the embodiment of the disclosure, the degree of close contact between the surface of the arm 50 and the trapezoidal prism 21 is confirmed, i.e., determined, on the basis of the output V2 of the second light-detecting element 25 in step S3 illustrated in FIG. 9, step S3A illustrated in FIG. 10, step S13 illustrated in FIG. 13, and step S13A illustrated in FIG. 14. According to the embodiment, the degree of close contact between the surface of the arm 50 and the trapezoidal prism 21 is confirmed, i.e., determined, by monitoring the output V2 of the second light-detecting element 25 based on the light coming from the single light-emitting element 20.

However, the degree of close contact between the surface of the arm 50 and the trapezoidal prism 21 may be confirmed, i.e., determined, on the basis of the output V2 of the second light-detecting element 25 by monitoring the output V of the second light-detecting element 25 based on light coming from a different light-emitting element 20 at a different time. With such an arrangement, the degree of close contact between the surface of the arm 50 and the trapezoidal prism 21 may be confirmed, i.e., determined, on the basis of light in a different wavelength range, for example, emitted from the different light-emitting element 20. Providing the multiple second light-detecting elements 25, e.g., the second light-detecting elements 25a and 25b illustrated in FIG. 15, detect the light coming from the multiple light-emitting elements 20, the outputs V2 of the second light-detecting elements 25 based on the light coming from the different light-emitting elements 20 may be monitored at different times or simultaneously. Providing the protrusive portions 26b and 26c (see FIG. 16) of the single second light-detecting element 26 detect the light coming from the multiple light-emitting elements 20, the outputs V2 of the second light-detecting elements 26 based on the light coming from the different light-emitting elements 20 may be monitored at different times.

The preferred embodiment and modifications according to the present disclosure have been described in detail above, it will be obvious to those skilled in the art that various changes, modifications, and revisions can be made therein without departing from the scope of the disclosure. Such changes, modifications, and revisions should fall within the scope of the disclosure.

The present disclosure contains subject matter related to that disclosed in Japanese Priority Patent Application JP 2025-042960 filed in the Japan Patent Office on Mar. 17, 2025, the entire content of which is hereby incorporated by reference.

What is claimed is:

1. A sensor device comprising:
a light-emitting element;
a prism that is of a trapezoidal contour as viewed along a direction and has a first bottom surface for contact with a surface of a human body, a second bottom surface lying parallel to the first bottom surface, and multiple slanted surfaces joining the first bottom surface and the second bottom surface to each other, the prism refracting light emitted from the light-emitting element and applied to a first slanted surface among the multiple slanted surfaces toward the human body;
a first light-detecting element that detects, of the light emitted from the light-emitting element, light that has been reflected by a region in the human body, refracted by the prism, and emitted from the prism;
a second light-detecting element that detects, of the light emitted from the light-emitting element, light that has been reflected by the first bottom surface of the prism into the prism and emitted from the prism;
an estimating section that estimates a concentration of a biological component included in the human body from an output from the first light-detecting element; and
a determining section that determines whether the estimating section is allowed to estimate the concentration of the biological component or not on a basis of at least an output from the second light-detecting element.

2. The sensor device according to claim 1, wherein
the determining section decides that the estimating section is allowed to estimate the concentration of the biological component if a magnitude of the output from the second light-detecting section is equal to or smaller than a first threshold value and that the estimating section is not allowed to estimate the concentration of the biological component if the magnitude of the output from the second light-detecting section is larger than the first threshold value.

3. The sensor device according to claim 2, further comprising:
an indicating section that gives an indication to a user of the sensor device, wherein
the determining section causes the indicating section to issue a request for prompting the user to press the prism strongly against the human body if the magnitude of the output from the second light-detecting section is larger than the first threshold value.

4. The sensor device according to claim 3, wherein
the determining section causes the indicating section to indicate that the estimating section cancels its operation to estimate the concentration of the biological component and causes the estimating section to cancel the operation if the magnitude of the output from the second light-detecting section is equal to or smaller than the first threshold value after elapse of a predetermined period of time from an initial event of detection that the magnitude of the output from the second light-detecting element is larger than the first threshold value.

5. The sensor device according to claim 2, further comprising:
a pressing mechanism that presses the prism against the human body; and
a controlling section that controls the pressing mechanism, wherein,
when the magnitude of the output from the second light-detecting section exceeds the first threshold value, the controlling section controls the pressing mechanism to increase its pressing force until the magnitude of the output from the second light-detecting section becomes equal to or smaller than the first threshold value, and after the magnitude of the output from the second light-detecting section has become equal to or smaller than the first threshold value, the controlling section maintains the pressing force, and
the estimating section estimates the concentration of the biological component from the output from the first light-detecting element under the maintained pressing force.

6. The sensor device according to claim 5, wherein
the controlling section
drives the pressing mechanism in a first mode during an initial stage of its control over the pressing mechanism, and,
after the initial stage, drives the pressing mechanism in a second mode that is of a lower rate than the first mode to increase the pressing force from the pressing mechanism until the magnitude of the output from the second light-detecting section becomes equal to or smaller than the first threshold value.

7. The sensor device according to claim 1, wherein
the determining section decides that the estimating section is allowed to estimate the concentration of the biological component if a ratio of the magnitude of the output from the second light-detecting element to the magnitude of the output from the first light-detecting section is equal to or smaller than a second threshold value and that the estimating section is not allowed to estimate the concentration of the biological component if the ratio is larger than the second threshold value.

8. The sensor device according to claim 7, further comprising:
an indicating section that gives an indication to a user of the sensor device, wherein
the determining section causes the indicating section to issue a request for prompting the user to press the prism strongly against the human body if the ratio is larger than the second threshold value.

9. The sensor device according to claim 8, wherein
the determining section causes the indicating section to indicate that the estimating section cancels its operation to estimate the concentration of the biological component and causes the estimating section to cancel the operation if the ratio is equal to or smaller than the second threshold value after elapse of a predetermined period of time from an initial event of detection that the ratio is larger than the second threshold value.

10. The sensor device according to claim 7, further comprising:
a pressing mechanism that presses the prism against the human body; and
a controlling section that controls the pressing mechanism, wherein,
when the ratio exceeds the second threshold value, the controlling section controls the pressing mechanism to increase its pressing force until the ratio becomes equal to or smaller than the second threshold value, and after the ratio has become equal to or smaller than the second threshold value, the controlling section maintains the pressing force; and
the estimating section estimates the concentration of the biological component from the output from the first light-detecting element under the maintained pressing force.

11. The sensor device according to claim 10, wherein
the controlling section
drives the pressing mechanism in a first mode during an initial stage of its control over the pressing mechanism, and,
after the initial stage, drives the pressing mechanism in a second mode that is of a lower rate than the first mode to increase the pressing force from the pressing mechanism until the ratio becomes equal to or smaller than the second threshold value.

12. The sensor device according to claim 1, wherein
the first light-detecting element is disposed to detect light emitted from the second bottom surface, and
the second light-detecting element is disposed to detect light emitted from a second slanted surface among the multiple slanted surfaces that is disposed opposite the first slanted surface across the second bottom surface.

13. The sensor device according to claim 1, wherein
the first light-detecting element is disposed to detect light emitted from the second bottom surface, and
the second light-detecting element is disposed to detect light emitted from the second bottom surface.

14. The sensor device according to claim 13, wherein
the second bottom surface of the prism has a central area and a peripheral area disposed in a periphery of the central area,
the first light-detecting element is disposed in facing relation to the central area, and
the second light-detecting element is disposed in facing relation to the peripheral area.

15. The sensor device according to claim 13, wherein
the second bottom surface of the prism has a central area and a peripheral area disposed in a periphery of the central area,
the first light-detecting element is disposed in facing relation to the central area, and
the second light-detecting element has an area larger than the first light-detecting element and has an overlapping portion overlapping the first light-detecting element and a protrusive portion protruding from the first light-detecting element, the protrusive portion being disposed in facing relation to the peripheral area.
